(19) United States
(12) Patent Application Publication
(10) Pub. No.: US 2024/0263122 A1
(43) Pub. Date: Aug. 8, 2024

(54) BIOREACTOR APPARATUS, ION-EXCHANGE SUBSTRATE AND METHOD FOR REGULATION OF ION CONCENTRATION AND CELL CULTURE THEREOF

(71) Applicants: National Central University, Taoyuan City (TW); National Defense Medical Center, Taipei City (TW)

(72) Inventors: Ching-Yun Chen, Taoyuan City (TW); Chih-Hung Wang, Taipei City (TW); Hsin-Chien Chen, Taipei City (TW); Yu-Hsuan Ting, Changhua County (TW); Chun-Yi Peng, Taoyuan City (TW); Yueh-Teng Tsai, New Taipei City (TW); Sheng-Wen Chang, Hsinchu City (TW); Feng-Yuan Chien, New Taipei City (TW)

(21) Appl. No.: 18/368,554

(22) Filed: Sep. 14, 2023

Related U.S. Application Data
(60) Provisional application No. 63/443,991, filed on Feb. 8, 2023.

(51) Int. Cl. C12M 1/00

(52) U.S. Cl. CPC C12M 29/04; C12M 29/14

(57) ABSTRACT

Provided is a bioreactor apparatus including: a liquid storage chamber for storing a liquid containing a first ion; a pump; a culture chamber for accommodating the liquid and cells to be cultured; and an ion-exchange chamber accommodating an ion-exchange substrate containing a second ion. Affinity of the second ion to the ion-exchange substrate is lower than affinity of the first ion to the ion-exchange substrate, or molar concentration of the second ion far outweigh molar concentration of the first ion. The storage chamber, the pump, the culture chamber and the ion-exchange chamber are connected via pipelines to form a closed loop, and the pump is configured to provide pressure to drive flow of the liquid in the closed loop. Also provided are a method for regulating ion concentration by the ion-exchange substrate and a method for cell culture by the bioreactor apparatus.

### CROSS REFERENCE TO RELATED APPLICATIONS

This application claims the benefit of U.S. Provisional Application No. 63/443,991, filed on Feb. 8, 2023. The content of the application is incorporated herein by reference.

### BACKGROUND OF THE INVENTION

### 1. Field of the Invention

The present disclosure relates to an ion-regulating technology, especially an ion-regulating technology applied to a culture solution.

### 2. Description of the Prior Art

Ion concentration in vivo may undergo dynamic changes due to external stimuli, developmental stages, cell-cell interactions, and other factors. However, in ex-vivo cultures, the ion concentration in the culture medium remains constant, making it difficult to dynamically regulate it in real-time to faithfully replicate in-vivo conditions. This leads to a significant disparity between the results of in-vitro and in-vivo experiments or adverse outcomes when cells cultured ex-vivo are transplanted back in-vivo owing to dynamic changes in ion concentration.

For instance, in the inner ear, endolymph initially consists of a high concentration of sodium ions (Na⁺) and a low concentration of potassium ions (K⁺) (referred to as high Na⁺/low K⁺) during embryonic development. However, as the embryo develops, the potassium ion concentration gradually increases to match that of sodium ions. After birth, potassium ions continue to increase, resulting in endolymph becoming a fluid with high potassium ions and low sodium ions (referred to as high K⁺/low Na⁺) (K⁺=154 mM; Na⁺=1 mM). Existing ex-vivo culture environments do not allow for gradual adjustment of ion concentrations to mimic the in-vivo state during cell growth. A Sudden fluctuation in ion concentrations in the ex-vivo culture microenvironment is detrimental to cell growth.

Hearing loss is a common ailment, and one of the primary causes is damage to hair cells in the inner ear, which serve as sensory cells, leading to an inability to regenerate and transmit neural signals to the brain. Currently, common clinical solutions for improvement involve cochlear implants; however, these cannot fully restore hearing. Therefore, the introduction of cell-based therapies for hearing loss has a great potential.

The endolymph within the cochlea undergoes transformation from high Na⁺/low K⁺ to high K⁺/low Na⁺ during embryonic development. However, the commonly used cell culture medium composition in ex-vivo cell cultures is high Nat/low Kt, which differs from the environment required for inner ear hair cell development and differentiation. This may result in differentiated inner ear hair cells that lack functionality, hindering cell-based therapies. Changing the culture medium from high Na⁺/low K⁺ to high K⁺/low Na⁺ in a conventional manner can lead to drastic changes in the cell culture environment, which is unfavorable for cell growth or differentiation. Furthermore, existing cell-based therapies typically employ two-dimensional (2D) cell culture, whereas three-dimensional (3D) cell culture is better at replicating cell-cell and cell-environment interactions. Cells obtained through 3D cell culture respond to stimuli from the local environment, making them more similar to the in-vivo environment.

Therefore, the development of biomimetic devices and ion control technologies to address these issues is a pressing challenge in this field.

### SUMMARY OF THE INVENTION

To address the aforementioned issues, the present disclosure provides a bioreactor apparatus comprising: a liquid storage chamber used for storing a liquid containing a first ion; a pump; a cultivation chamber for accommodating the liquid and a cell to be cultivated; and an ion exchange chamber containing an ion-exchange substrate comprising a second ion. Affinity of the second ion for the ion-exchange substrate is lower than affinity of the first ion to the ion-exchange substrate, or molar concentration of the second ion is higher than molar concentration of the first ion. The liquid storage chamber, the pump, the cultivation chamber, and the ion exchange chamber are interconnected by a pipeline to form a closed loop, and the pump is configured to provide pressure to drive the liquid to flow within the closed loop.

The present disclosure provides a method for regulating ion concentration in a liquid, comprising: providing an ion-exchange substrate containing a second ion and having a semi-permeable membrane enclosing a liquid comprising the second ion; and disposing the ion-exchange substrate in the culture medium comprising a first ion to perform ion exchange, wherein affinity of the second ion to the ion-exchange substrate is lower than affinity of the first ion to the ion-exchange substrate, or molar concentration of the second ion is higher than molar concentration of the first ion. The ion exchange comprises: allowing the first ion to enter the ion-exchange substrate through the semi-permeable membrane; and releasing the second ion from the ion-exchange substrate into the liquid through the semi-permeable membrane.

The present disclosure further provides a method for culturing a cell, comprising: providing the bioreactor apparatus of the present disclosure; disposing the culture medium and the cell in a cultivation chamber, driving the culture medium to flow in the closed loop using a pump, and performing ion-exchange using the ion-exchange substrate to decrease the concentration of the first ion and increase the concentration of the second ion in the culture medium.

In at least one embodiment of the present disclosure, the liquid further comprises the second ion, and during ion exchange, the concentration of the second ion in the ion-exchange material is greater than the concentration of the second ion in the liquid; after ion exchange, the concentration of the first ion in the liquid decreased, and the concentration of the second ion in the liquid increased.

In at least one embodiment of the present disclosure, the ion-exchange substrate comprises a semi-permeable membrane encapsulating the liquid containing the second ion, allowing the first ion to enter the ion-exchange substrate and release the second ion into the liquid.

In at least one embodiment of the present disclosure, the ion-exchange substrate is formed from a crosslinked construct of an alginate and a third ion, and the affinity of the third ion to the ion-exchange substrate is greater than affinity of the second ion for the ion-exchange substrate.

In at least one embodiment of the present disclosure, the first ion comprises Na⁺, NH₄⁺, or a heavy metal ion; the second ion comprises Na⁺, K⁺, Ca²⁺, or NH₄⁺; and the third ion comprises a divalent cation, a trivalent, or a polyvalent cation. In some embodiments, the present disclosure can be used to improve water quality by adsorbing heavy metal ions from wastewater to achieve wastewater treatment objectives. In some embodiments, the heavy metal ion is at least one selected from a group consisting of Cd²⁺, Pb²⁺, Cr²⁺, Sb²⁺, Hg²⁺, Fe²⁺, Co²⁺, Zn²⁺, Ni²⁺, Cu²⁺, Tb³⁺, Dy³⁺, Fe³⁺, and Al³⁺. In some embodiments, the divalent cation is at least one selected from a group consisting of Sr²⁺, Ca²⁺, Cd²⁺, Pb²⁺, Cr²⁺, Ba²⁺, Se²⁺, Sb²⁺, Hg²⁺, Mg²⁺, Fe²⁺, Co²⁺, Zn²⁺, Ni²⁺, As²⁺, and Cu²⁺. In some embodiments, the trivalent cations is at least one selected from a group consisting of Tb³⁺, Dy³⁺, Fe³⁺, and Al³⁺. In some embodiments, the third ion is Sr²⁺, Ca²⁺, or Mg²⁺. In some embodiments, the second ion is K⁺, and the third ion is Sr²⁺. In at least one embodiment of the present disclosure, the first ion is Na⁺, NH₄⁺, heavy metal ion, or any combination thereof, and the second ion is K⁺.

In at least one embodiment of the present disclosure, the crosslinked construct forms a cavity having the semi-permeable membrane.

In at least one embodiment of the present disclosure, the crosslinked construct is in the form of particles or spheres. In some embodiments, the diameter of the particles or spheres ranges from 0.5 mm to 50 mm.

In at least one embodiment of the disclosed method, the cell is a hair cell of an inner ear.

In summary, the bioreactor apparatus and the ion exchange substrate of the present disclosure not only address the issues in prior art related to in-vitro cell culture, but also provide the following features and effects in certain embodiments:
(a) Real-time control of ion concentrations in the culture medium during in-vitro cell cultivation to faithfully replicate in-vivo conditions.
(b) Provision of a 3D cell culture approach, simulating cellular interactions and cellular-environment interactions within a living organism, thus facilitating cellular responses to stimuli from the environment.
(c) Gradual and sequential adjustments that dynamically modify ion concentrations in the microenvironment of in-vitro cell culture, aligning with the states observed during the growth process of cells within a living organism.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure will become more readily appreciated by reference to the following descriptions in conjunction with the accompanying drawings.

FIG. 1 is a schematic diagram illustrating a flowchart depicting the process for preparing potassium-alginate hydrogel beads (referred to as the K⁺-beads) according to the specific embodiment disclosed herein.

FIG. 2-1 is a schematic diagram illustrating a bioreactor apparatus as disclosed in one specific embodiment.

A to E in FIG. 2-2 are schematic depictions of the internal structure of a cultivation chamber in one specific embodiment of this disclosure.

FIG. 2-3A and FIG. 2-3B show schematic depictions of various configurations of the culture-dish holder in one specific embodiment of this disclosure.

A to D in FIG. 3 are comparative diagrams showing the ion-exchange effects of the K⁺-beads with varying composition ratios in one specific embodiment.

FIG. 4 displays a line chart illustrating the variation in K⁺ (shown by a blue line), Na⁺ (shown by a red line) and total⁺¹ ion (shown by a gray line) concentrations of an improved culture medium utilizing the K⁺-beads for ion exchange, along with photographs showing the physical appearance of the beads after multiple ion-exchange processes in one specific embodiment of this disclosure.

A to C in FIG. 5 are comparative photographs of the K⁺-beads stored in different solutions/preservatives, as disclosed in one specific embodiment of this disclosure.

A to F in FIG. 6 are schematic representations and effect diagrams of the removal of nitrogen-containing waste using the K⁺-beads according in one specific embodiment of this disclosure.

A and B in FIG. 7 are schematic diagrams of the ion exchange of the dynamic circulation system within the bioreactor in one specific embodiment of this disclosure.

FIG. 8 shows a comparative chart of cell growth morphology when cultivating cells in a medium modified by the K⁺-beads versus DMEM-HG medium, as disclosed in one specific embodiment of this disclosure.

FIG. 9 shows a bar chart illustrating the biocompatibility test results of the K⁺-beads, according to one specific embodiment of this disclosure.

FIG. 10 shows the cell growth and differentiation patterns of murine cochlear progenitor cells (CPC) cultivated using a medium modified by the K⁺-beads, as disclosed in one specific embodiment of this disclosure.

FIG. 11 provides a comparative chart showing the growth and differentiation patterns of murine CPC clusters cultivated in the culture medium with different ion concentrations, as disclosed in one specific embodiment of this disclosure.

A and B in FIG. 12 present a schematic diagram illustrating the immunocytochemical characteristics of murine CPC cultivated in a medium modified by the K⁺-beads in one specific embodiment of this disclosure.

FIG. 13 depicts a trend chart displaying changes in the cell size of murine CPC clusters cultivated in a medium modified by the K⁺-beads according to one specific embodiment of this disclosure.

FIG. 14 shows a viability assay diagram measuring cell vitality (live/dead) of murine cochlear progenitor cells cultivated in a medium modified by the K⁺-beads in one specific embodiment of this disclosure.

FIG. 15 shows the immunocytochemical characteristics of the 3D-bioprinted mouse CPC-laden scaffold in a culture-dish holder in one specific embodiment of this disclosure.

### DETAILED DESCRIPTION

The following descriptions of the embodiments illustrate implementations of the present disclosure, and those skilled in the art of the present disclosure can readily understand the advantages and effects of the present disclosure in accordance with the contents herein. However, the embodiments of the present disclosure are not intended to limit the scope of the present disclosure. The present disclosure can be practiced or applied by other alternative embodiments, and every detail included in the present disclosure can be changed or modified in accordance with different aspects and applications without departing from the essentiality of the present disclosure.

The present disclosure provides specific exemplary embodiments to illustrate the implementation of the disclosed subject matter within the field of technology, which are readily understood by those skilled in this art based on the contents disclosed herein, including the essence, advantages, and benefits of the present disclosure. However, the specific embodiments described herein are not intended to limit the present disclosure, as it can be implemented or applied through other different embodiments, and the details provided in the present disclosure can be subject to various modifications from different perspectives and applications, all within the spirit of the present disclosure.

The proportions, structures, sizes, and other features shown in the accompanying drawings are used solely to complement the content disclosed herein for the understanding of those skilled in the art in the field of the present disclosure and not to limit the scope of implementation of the present disclosure. Therefore, any changes in proportional relationships, structural modifications, or size adjustments that do not affect the objectives and effects achievable by the present disclosure should be within the scope of the technical content disclosed in the present disclosure.

When terms such as “comprising.” “including,” or “having” specific elements are used in the present disclosure, unless otherwise specified, they may also include other elements, components, structures, regions, parts, devices, systems, steps, or interconnections, and do not exclude such other elements.

As used in the present disclosure, terms such as “up,” “down,” “left.” “right.” “inside.” “outside.” and the like are used for explanatory purposes regarding the implementation of the present disclosure and are not intended to limit the scope of the present disclosure. Any adjustments, substitutions, or changes in relative positions and relationships that do not substantially change the technical content of the present disclosure should be within the scope of its protection.

The terms “first.” “second.” “third.” and the like used in the present disclosure are for the purpose of describing or distinguishing elements, components, structures, regions, parts, devices, systems, and the like and are not used to limit the scope of implementation of the present disclosure, nor are they used to specify the spatial order of such elements. Furthermore, unless otherwise expressly stated in the present disclosure, the singular form “a” and “the” also include the plural form, and the term “or” can be used interchangeably with “and/or.”

The numerical ranges described in the present disclosure are inclusive and combinable. Any value within the numerical range can be used as either the maximum or minimum value to derive the subranges. For example, the numerical range “0.5 mm to 50 mm” should be understood to encompass any subrange between the minimum value of 0.5 mm and the maximum value of 50 mm, such as 0.5 mm to 5 mm, 2 mm to 10 mm, and 2.5 mm to 3.5 mm, among others. Additionally, the multiple numerical endpoints described in the present disclosure can be arbitrarily selected as the maximum or minimum values to derive numerical ranges. For example, 0.5, 5, and 50-mm yielded the numerical ranges of 0.5 to 5 mm, 5 to 50 mm, or 0.5 to 50 mm, respectively.

The term “connection” used in the present disclosure refers to the joining of multiple components directly or indirectly, where “direct connection” means the direct contact and joining of multiple components, and “indirect connection” means joining multiple components through at least one connecting element. Means of achieving “connection” in the present disclosure include close magnetic attraction, dovetail joints, connectors, pivots, linkages, sutures, adhesion, embedding, screwing, snapping, nailing, clamping, affixing, threading, gripping, positioning, integral forming, or combinations thereof. In at least one specific embodiment of the present disclosure, multiple components are “removably” connected, meaning that they can be separated from each other after being connected. Furthermore, the term “connecting element” in the present disclosure refers to an element that facilitates the means of “connection” described above.

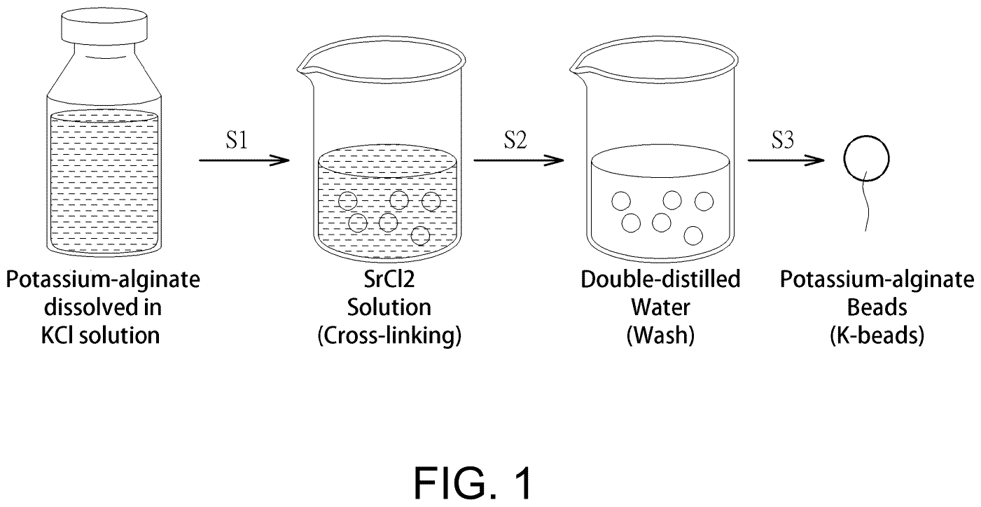

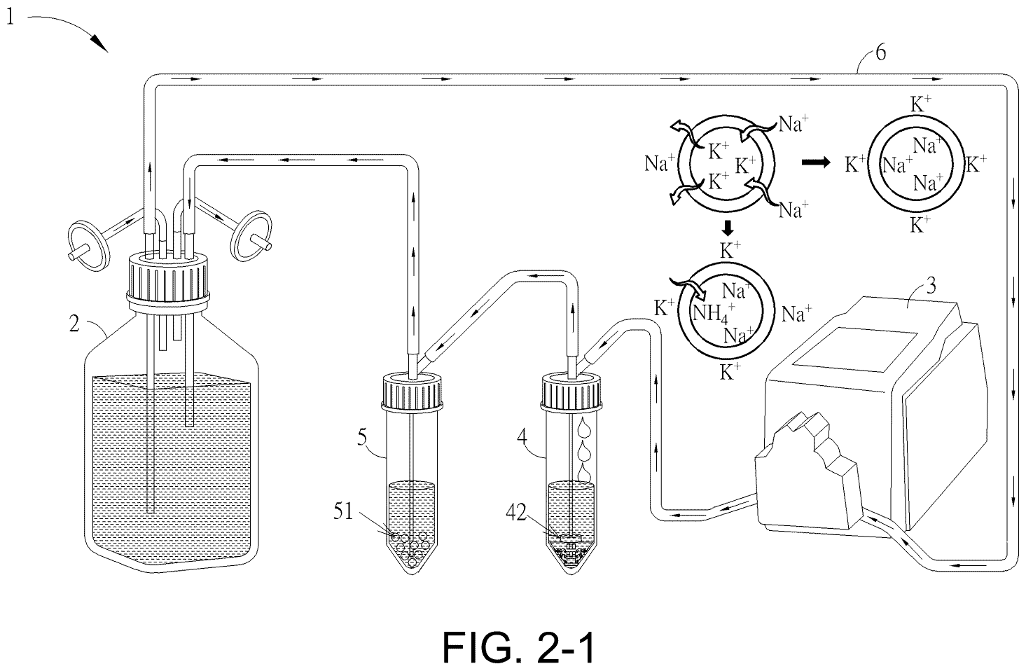

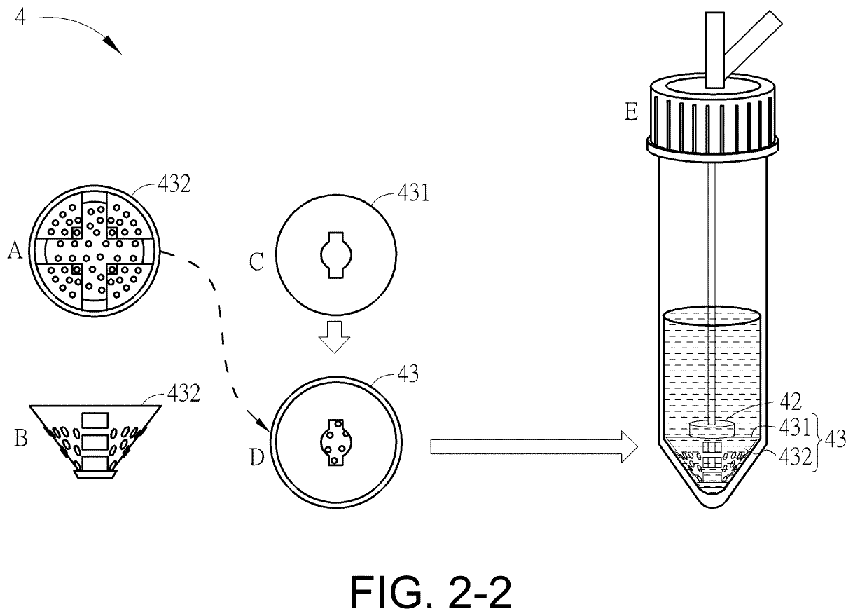

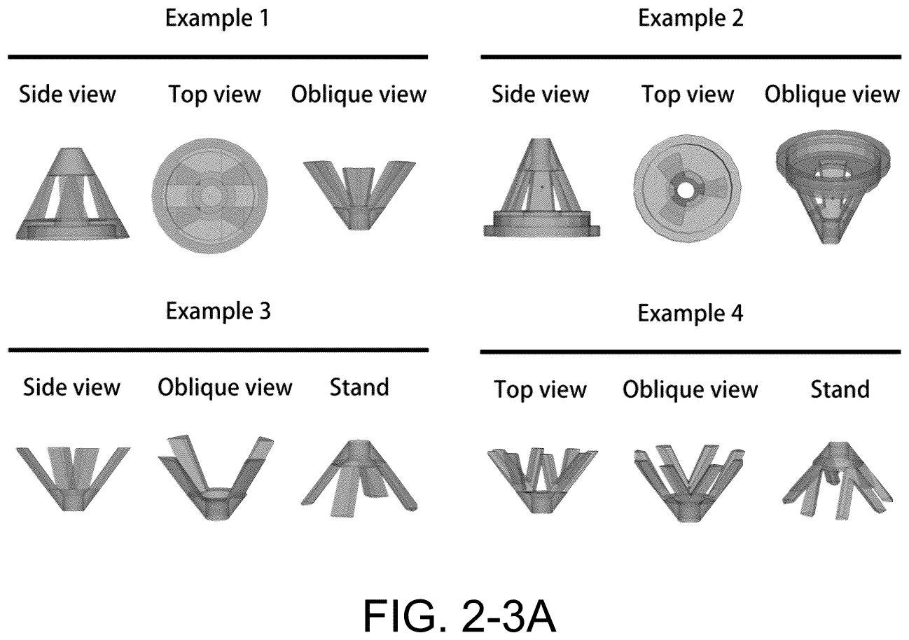

In at least one specific embodiment of the present disclosure, the fluid storage chamber, pump, cultivation chamber, and ion-exchange chamber are interconnected by pipelines to form a closed loop. The pump is configured to provide pressure to drive the fluid to flow in a closed loop. Multiple cultivation chambers can be used, and these multiple cultivation chambers are interconnected via pipelines. Therefore, cultivation fluid can serve as a medium for signal transmission. For example, the cultivation fluid from the first cultivation chamber can carry substances secreted by cells to flow into the second cultivation chamber, thereby allowing these substances to influence the cells being cultured in the first cultivation chamber. Through this mechanism, the disclosed bioreactor apparatus can simulate the signal transmission within a biological organism. As shown in FIG. 2-1 of the present disclosure, only one cultivation chamber 4 is shown. However, the user can adjust the number of cultivation chambers 4 as needed, and cultivation chamber 4 can be interconnected in series, parallel, or in combination to better mimic the real conditions within a biological organism.

The term “hearing loss” used in the present disclosure can be divided into three main categories: conductive hearing loss, sensorineural hearing loss, and mixed hearing loss. Conductive hearing loss may result from structural defects or abnormalities in the car, such as car canal closure, incomplete development of the outer car, obstruction by foreign bodies, such as carwax, or defects in the auditory ossicles of the middle car. Additionally, conductive hearing loss can occur because of middle car effusion caused by middle ear infections. Sensorineural hearing loss is caused by damage to the inner ear, vestibulocochlear nerve, or hair cells in the central processing centers of the brain. Since hair cells cannot regenerate and current treatment methods cannot effectively repair sensorineural hearing loss, this remains a challenging problem.

The term “hair cell regeneration” used in the present disclosure is divided into two main categories: (1) support cells surrounding hair cells re-enter the cell cycle after mitosis (including the cell growth phase and mitotic phase) to differentiate into hair cells, and (2) support cells directly transdifferentiate into hair cells. Currently, three main types of stem cells are used for hair cell regeneration: tissue-specific progenitor, mesenchymal, and induced pluripotent stem cells.

The term “2D cell culture” in the present disclosure refers to the cultivation of cells on a flat surface, ensuring that all cells on the surface have equal and sufficient access to nutrients and air. Since the growth and differentiation rates of most cells are relatively similar, 2D cell culture is operationally simpler and allows for mass cultivation of cells. However, the disadvantage of 2D cell culture is that the cellular environment for growth lacks interactions between cells, and between cells and the environment, making it different from in-vivo cell growth conditions. Furthermore, cells in 2D cell culture grow in an adherent manner, resulting in cell configurations different from those in the body, and therefore cannot be used for cell therapy. In recent years, regenerative medicine and in-vitro platforms that mimic the circulatory system of living organisms have emerged, and 2D cell culture is inadequate to meet these needs, leading to the development of 3D cell culture.

The term “3D cell culture” used in the present disclosure refers to the cultivation of cells in a three-dimensional environment, such as in hydrogels or biocompatible materials like alginate, to mimic the growth environment in-vivo. During cultivation, cells synthesize and secrete the extracellular matrix (ECM), which forms clusters and shapes the three-dimensional cell growth environment. ECM plays an important role in cell culture, assisting with cell growth, movement, and other functions. It can also aid in cell adhesion and communication between cells. Additionally, cells differentiate and grow to varying degrees at different locations within the clusters, creating interactions between cells. Therefore, cells cultured in a three-dimensional environment more closely resemble those existed in-vivo.

The term “alginate” used in the present disclosure is a natural polymer composed of repeating units of α-L-guluronate extracted from sources such as brown algae or kelp. Sodium-alginate is the most widely used type of alginate in various applications; however, alginate types include, but are not limited to, ammonium-alginate, calcium-alginate, potassium-alginate, and other salt compounds. Different types of alginates have different properties, such as solubility and gel-forming characteristics. Alginate is non-toxic, readily available in large quantities, biodegradable, and biocompatible, making it a common material for biomedical applications. Alginate consists of β-D-mannuronic acid (M-block) and α-L-guluronic acid (G-block) monomers, and its composition includes random segments such as poly-MM, poly-GG, or poly-MG. The stereoisomeric structure of alginate leads to electrostatic repulsion, resulting in different rigidities for various random combinations. Additionally, the proportion and distribution of M-blocks and G-blocks in alginate extracted from different sources may vary, allowing users to select appropriate combinations as needed.

In some embodiments of the present disclosure, alginate forms hydrogels by cross-linking with monovalent, divalent, or higher-valent cations. The affinity sequence of divalent and other non-divalent cations with alginate is as follows: Sr2+>Pb2+>Tb3+>Dy3+>Ca2+>Cd2+>Mg2+>Fe2+>Fe3+>Co2+>A13+>Ni2+>Cs2+>Cu2+>Ag+>Li+>Na+>K+.

The term “cross-linking” used in the present disclosure refers to the mutual cross-linking of numerous molecules (usually linear or lightly branched molecules) to form a more stable three-dimensional network structure. In some embodiments disclosed herein, alginate is a viscous liquid that undergoes cross-linking with divalent or higher-valent metal cations to transform into a transparent and elastic hydrogel. However, the characteristics of the hydrogel described in this work are influenced by the type of alginate, type of salt, valency, ionic radius, presence of chelating agents, temperature, and pH. In some embodiments disclosed herein, when an alginate solution (such as sodium alginate) is dripped into a solution containing divalent or higher-valent metal cations (such as calcium chloride), the calcium ions replace Na+ on the carboxyl groups of sodium alginate and bind to the carboxyl groups of another alginate molecule, forming a three-dimensional network structure (i.e., the hydrogel). This strengthens the connections between sodium alginate molecules, and the gel can encapsulate contents within its structure, forming the semipermeable membrane described in this work, which releases contents in specific environments (i.e., the ion-exchange environment described in this work).

In some embodiments of the present disclosure, alginate cross-links with divalent cations such as Ca2+ to form alginate containing calcium ions. The concentration of Ca2+ in the intralymphatic fluid is low (approximately 20 to 30 μM), and maintaining this concentration is necessary for normal hearing, as Ca2+ regulates various hair cell functions. Therefore, in the other embodiments of the present disclosure, alginate cross-links with divalent cations such as Sr2+ because of its higher affinity for alginate compared to other ions. This makes it less likely to be replaced by other ions in the culture medium, thereby affecting ion balance in the medium. Additionally, during the neurotransmitter release process controlled by Ca2+ at the neuromuscular junction, Sr2+ can serve as a substitute for Ca2+. Therefore, even if a small amount of Sr2+ dissociates from the alginate complex, it does not affect cell function.

The term “crosslinked construct,” as used in the present disclosure, refers to any cavity containing a second ion within a semi-permeable membrane. In at least one specific embodiment of the present disclosure, the shape of the crosslinked construct includes, but is not limited to, particles, spheres, discoidal spheres, elongated bodies, triangles, cubes, rectangular prisms, cylinders, cones, ellipsoids, ovoids, triangular pyramids, triangular prisms, tetrahedra, quadrangular pyramids, irregular shapes, or any combination thereof.

In some embodiments of the present disclosure, the particle size of the crosslinked construct can range from 0.5 mm to 50 mm, 5 mm to 50 mm, 10 mm to 50 mm, 15 mm to 50 mm, 20 mm to 50 mm, 25 mm to 50 mm, 30 mm to 50 mm, 35 mm to 50 mm, 40 mm to 50 mm, 45 mm to 50 mm, 0.5 mm to 45 mm, 5 mm to 45 mm, 10 mm to 45 mm, 15 mm to 45 mm, 20 mm to 45 mm, 25 mm to 45 mm, 30 mm to 45 mm, 35 mm to 45 mm, 0.5 mm to 40 mm, 5 mm to 40 mm, 10 mm to 40 mm, 15 mm to 40 mm, 20 mm to 40 mm, 25 mm to 40 mm, 30 mm to 40 mm, 35 mm to 40 mm, 0.5 mm to 35 mm, 5 mm to 35 mm, 10 mm to 35 mm, 15 mm to 35 mm, 20 mm to 35 mm, 25 mm to 35 mm, 30 mm to 35 mm, 0.5 mm to 30 mm, 5 mm to 30 mm, 10 mm to 30 mm, 15 mm to 30 mm, 20 mm to 30 mm, 25 mm to 30 mm, 0.5 mm to 25 mm, 5 mm to 25 mm, 10 mm to 25 mm, 15 mm to 25 mm, 20 mm to 25 mm, 0.5 mm to 20 mm, 5 mm to 20 mm, 10 mm to 20 mm, 15 mm to 20 mm, 0.5 mm to 15 mm, 5 mm to 15 mm, 10 mm to 15 mm, 0.5 mm to 10 mm, 5 mm to 10 mm, and 0.5 mm to 5 mm, but the present disclosure is not limited thereto.

In at least one specific embodiment of the present disclosure, the alginate used in this art is potassium alginate, and ion exchange is performed in the culture medium using different ions with varying affinities for potassium alginate. This results in the formation of potassium-alginate hydrogel beads (the K+-beads) with semipermeable properties owing to cross-linking. Potassium chloride (KCl) is added to the K+-beads, which are then placed in culture medium for ion exchange. This creates a microenvironment of high K+ and low Na+ in the culture medium, establishing an ex-vivo ion replacement and regulation technique.

In some embodiments of the present disclosure, the ion exchange and regulation technique described in this art can be applied not only to the growth, differentiation, and maturation of cochlear progenitor cells (CPC) but also to other in-vitro models with ion regulation requirements, without limitations.

In some embodiments of the present disclosure, potassium-alginate adsorbs NH₄+ and can remove nitrogen-containing substances from aqueous solutions.

In some embodiments of the present disclosure, the K+-beads in the bioreactor apparatus described in this art can not only exchange Na+ and K+ in the culture medium but also adsorb nitrogen-containing metabolic wastes produced by the cells in the system, providing a more suitable environment for cell growth

In some embodiments of the present disclosure, the ability of potassium-alginate to release K+ is not affected by environmental pH. Potassium-alginate can decompose in the gastrointestinal tract into alginate and K+, and the separated alginate can form sodium-alginate with Na+ in the body and be excreted, thereby reducing the body's Na+ concentration. Therefore, potassium-alginate may improve hypernatremia.

The following is a further explanation of the features and benefits of the present disclosure using specific preparation examples and embodiments; however, it is not intended to limit the scope of the present disclosure.

### Preparation Example 1: Preparation of Cochlear Progenitor Cells (CPC)

CPC were isolated from the inner ear of neonatal mice at postnatal Day 0-2 (P0-P2) using the following steps: (I) Humane sacrifice of neonatal mice aged 0-2 days, followed by extraction of the mouse heads and removal of brain tissue and other tissues to leave behind the cochlear samples; (II) the cochlear samples were minced into a paste and digested with a digestive enzyme at 37° ° C. for 15 min, and the tissue was passed through a 70-μm filter to remove undissociated tissue; (III) the CPC within the tissue were dissociated into single cells and cultured in a 24-well cell culture plate, with a cell density of 1 cochlear CPC per well, and the days in-vitro (DIV) were recorded; and (IV) after 24 h, the suspended cells were transferred to a new 24-well cell culture plate. The suspended cell aggregates constituted the CPC.

### Preparation Example 2: Preparation of Potassium-Alginate Solution and Hydrogel Beads (K+-Beads)

### Preparation Example 2-1: Preparation of Potassium-Alginate Aqueous Solution

The concentration of potassium-alginate solution used in the present disclosure ranged from 1.5 wt % to 3.0 wt %. A 0.22-μm filter was used to filter 1.5 wt % to 2.0 wt % potassium-alginate solutions to remove bacteria and other impurities. Solutions with concentration above 2.0 wt % were too viscous to pass through the filter and were sterilized using an autoclave under high-temperature and high-pressure conditions.

In some embodiments of the present disclosure, potassium-alginate solutions may have a concentration ranging from 0.5 wt % to 5.0 wt %, 1.0 wt % to 5.0 wt %, 1.5 wt % to 5.0 wt %, 2.0 wt % to 5.0 wt %, 2.5 wt % to 5.0 wt %, 3.0 wt % to 5.0 wt %, 3.5 wt % to 5.0 wt %, 4.0 wt % to 5.0 wt %, 4.5 wt % to 5.0 wt %, 0.5 wt % to 4.5 wt %, 1.0 wt % to 4.5 wt %, 1.5 wt % to 4.5 wt %, 2.0 wt % to 4.0 wt %, 2.5 wt % to 4.5 wt %. 3.0 wt % to 4.5 wt %, 3.5 wt % to 4.5 wt %, 4.0 wt % to 4.5 wt %, 0.5 wt % to 4.0 wt %, 1.0 wt % to 4.0 wt %, 1.5 wt % to 4.0 wt %, 2.0 wt % to 4.0 wt %, 2.5 wt % to 4.0 wt %, 3.0 wt % to 4.0 wt %, 3.5 wt % to 4.0 wt %, 0.5 wt % to 3.5 wt %, 1.0 wt % to 3.5 wt %. 1.5 wt % to 3.5 wt %, 2.0 wt % to 3.5 wt %, 2.5 wt % to 3.5 wt %, 3.0 wt % to 3.5 wt %, 0.5 wt % to 3.0 wt %, 1.0 wt % to 3.0 wt %, 1.5 wt % to 3.0 wt %, 2.0 wt % to 3.0 wt %, 2.5 wt % to 3.0 wt %, 0.5 wt % to 2.5 wt %, 1.0 wt % to 2.5 wt %, 1.5 wt % to 2.5 wt %, 2.0 wt % to 2.5 wt %, 0.5 wt % to 2.0 wt %, 1.0 wt % to 2.0 wt %, 1.5 wt % to 2.0 wt %, and 0.5 wt % to 1.5 wt %, but the disclosure is not limited to these ranges.

### Preparation Example 2-2: Preparation of Potassium-Alginate Hydrogel Beads (K+-Beads)

As shown in FIG. 1, potassium-alginate solutions ranging from 0.5 wt % to 5.0 wt % were prepared by adding potassium chloride powder. These solutions were then dripped into a strontium chloride solution for cross-linking (S1). The crosslinked K+-beads were retrieved and washed with deionized water to remove residual strontium chloride (S2), and excess liquid was removed using paper towels (S3). The crosslinked K+-beads appeared as white semi-transparent spheres with a volume of approximately 25 μl and an average diameter of 3.21±0.07 mm (n=10).

### Preparation Example 2-3: Replacement of Na+ and K+ in Culture Medium Using K+-Beads

To analyze the ion replacement properties of the K+-beads s, the method for replacing Na+ and K+ in the culture medium included the following steps: (I) Placing the K+-beads prepared in Example 2-2 into a first centrifuge tube and adding the culture medium (bead volume:medium volume=1:2). The tube was then placed in a 37° C. incubator for 10 min, and (II) the culture medium was removed from the centrifuge tube and transferred to a second centrifuge tube containing K+-beads. This process was repeated until the Na+ and K+ in the culture medium were similar to those in mouse cochlear progenitor cells around the fifth day of birth (K+=154 mM; Na+=1 mM). After the K+-beads were exposed to the culture medium, their appearance changed from white semi-transparent to pink, and as the ion replacement process proceeded in the K+-beads, the color of the culture medium gradually became lighter. This was used to determine whether the semi-permeable membrane of the beads allowed phenol red from the culture medium to diffuse into the interior of the K+-beads.

### Preparation Example 3: Design and Assembly of Bioreactor Apparatus 1

As shown in FIG. 2-1, Bioreactor apparatus 1 consists of a liquid storage chamber 2, a cultivation chamber 4, an ion exchange chamber 5, and a pump 3, all interconnected by silicone tubing. This device is placed in a 37° C. incubator with 5% CO2. The design of the device involves the following steps: (I) culturing cochlear progenitor cells in the cultivation chamber 4; (II) placing the K+-beads in the ion-exchange chamber 5 for dynamic ion exchange between the K+-beads and the culture medium/liquid (i.e., release of K+ from the K+-beads and absorption of Na+ from the culture medium); and (III) using the pump 3 to pump liquid between the liquid storage chamber 2, cultivation chamber 4, and ion exchange chamber 5 at a flow rate of 1 ml/min to continuously replace the culture medium in the cultivation chamber 4, transitioning the environment within Bioreactor apparatus 1 from high Na+ to low K+. Additionally, above the liquid storage chamber 2, there are inlet and outlet tubes for maintaining pH stability within the system.

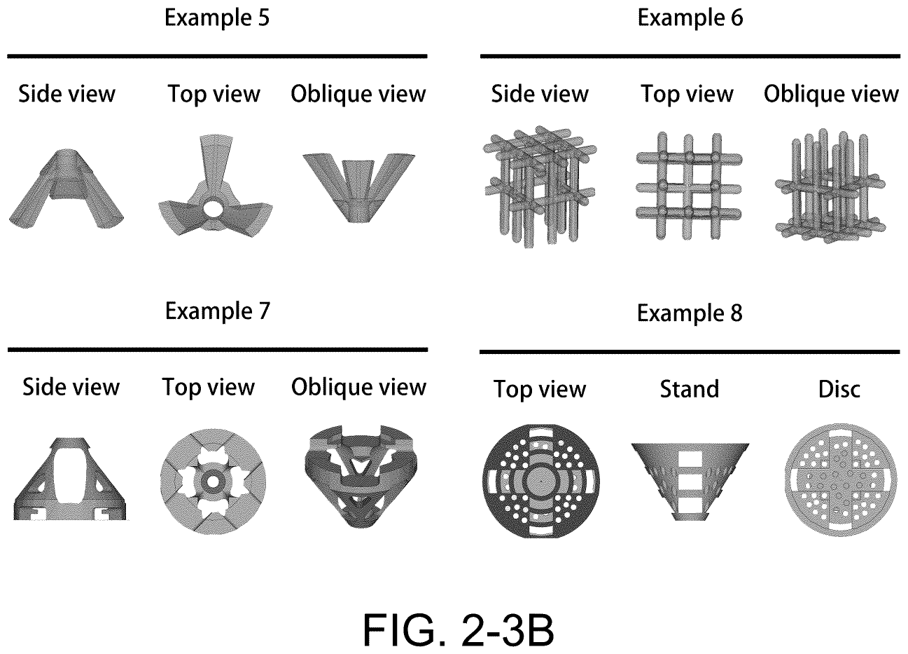

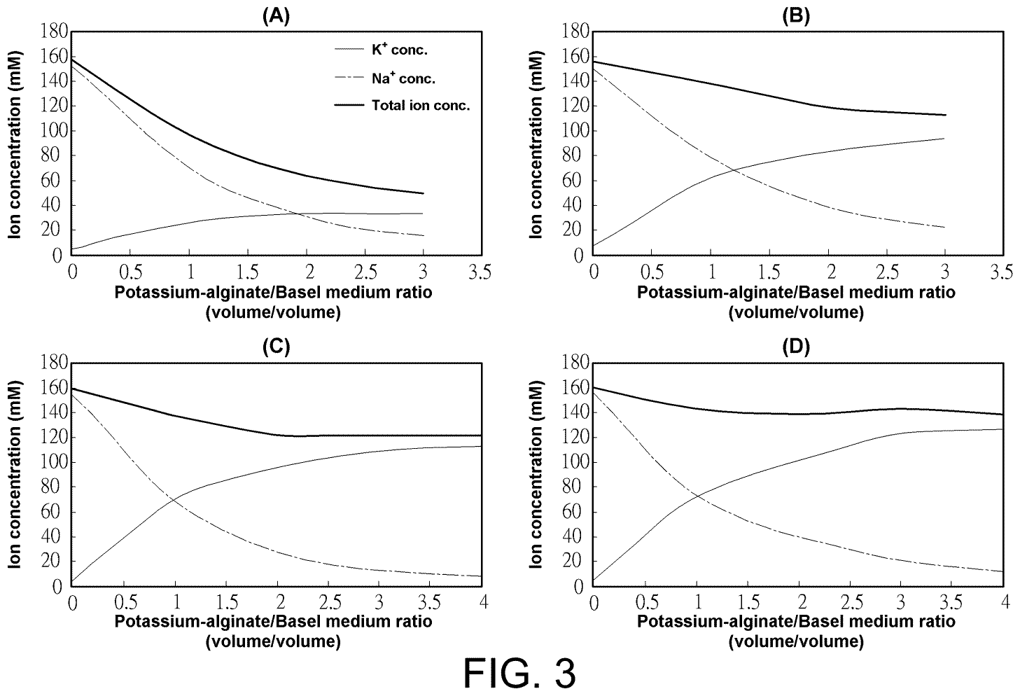

As shown in A to E in FIG. 2-2, FIG. 2-3A, and FIG. 2-3B, the bottom of cultivation chamber 4 (FIG. 2-2E) is equipped with a dish holder 43, consisting of a base 432 (A and B in FIG. 2-2) and a lid 431 (C in FIG. 2-2). FIG. 2-2A provides a top-down view of base 432 and FIG. 2-2B offers a side view of the same base 432. The shape of the cultivation dish holder 43 may include, but is not limited to, a bowl shape (patterns one to four in FIG. 2-3A; patterns five to eight in FIG. 2-3B). As depicted in A to E in FIG. 2-2, the bottom of cultivation chamber 4 is equipped with scaffold 42 (e.g., alginate, gelatin, collagen, chitosan, hyaluronic acid, methyl cellulose, chondroitin-sulfate, hydroxyapatite, Collgel®, etc.; however, the present disclosure is not limited thereto) to serve as a culture substrate for CPC in the disclosed dynamic perfusion system. The shape of scaffold 42 may include, but is not limited to, a ring shape. Specifically, the scaffold 42 is positioned on top of the cultivation dish holder 43 (D in FIG. 2-2: top-down view).

### Preparation Example 4: Preparation and Experimental Procedure Design for Biocompatibility Testing in Example 6

To test the biocompatibility of the K+-beads described in the present disclosure, L929 cells were used as test cells. The biocompatibility test was divided into four groups: the test group (containing culture medium extract with a test substance at 0.2 g/ml), the control group (culture medium with 10% FBS), the positive control group (culture medium with 10% DMSO and 10% FBS), and the negative control group (culture medium with HDPE film and 10% FBS). The cells were seeded at 10,000 cells/well in a 96-well cell culture plate and incubated at 37° C. for 24 h. The culture medium from each group was then added for 24 h. After removing the culture medium, the cells were washed with PBS and a CCK-8 (cell counting kit-8 assay) was performed. The absorbance values were measured using a microplate spectrophotometer after two hours later to estimate the number of viable cells.

### Embodiment 1: Ion Exchange Characteristics of the K+-Beads

### Embodiment 1-1: A Comparative Study of Ion Exchange Effects in the K+-Beads Treated by High-Temperature High-Pressure or Membrane Filtration Sterilization

Because the ion exchange process is achieved by direct interaction with the culture medium, the K+-beads require sterilization through either filtration or sterilization under high-temperature, high-pressure conditions. The viscosity of the potassium-alginate solution was higher than that of the sodium-alginate solution, and if the concentration of potassium alginate solution exceeds 1.5%, it cannot be filtered using a 0.22-μm membrane, necessitating high-temperature high-pressure sterilization of the potassium alginate solution. However, the high-temperature high-pressure sterilization method may lead to chemical degradation of the potassium alginate solution. Therefore, it is necessary to compare the ion-exchange stability of the K+-beads prepared from potassium alginate solutions treated by membrane filtration or high-temperature, high-pressure sterilization.

As shown in Table 1, the K+-beads of potassium alginate treated with 0.22-μm membrane filtration formed fragile semi-permeable membranes under the same crosslinking time because of the lower potassium-alginate concentration. During ion exchange, a large quantity of water molecules from the culture medium penetrates the bead structure, affecting the osmotic pressure of the culture medium. This leads to slower sodium ion exchange and faster release of potassium ions owing to their higher activity, necessitating a longer reaction time to reach ion equilibrium. On the other hand, the K+-beads of potassium alginate treated with high-temperature high-pressure sterilization in an autoclave exhibit a stronger semi-permeable membrane, achieving stable ion-exchange effects in a shorter time under the same crosslinking time. Moreover, they maintain the osmotic pressure of the culture medium. From this, it can be inferred that while the high temperature within the sterilization vessel may lead to the cleavage of polymer chains, it does not impact the ion exchange capability of the K+-beads disclosed in this work.

TABLE 1
The impact of different sterilization
methods on concentration replacement.

| | Sterilization Methods | | | |
|---|---|---|---|---|
| | 0.22-μm Membrane | | | |
| | Filtration | | Altoclave | |
| Concentration of | 1.5% | | 2.5% | |
| Potassium-alginate | | | | |
| Ion-exchange | 30 min | 24 hr | 30 min | 24 hr |
| Duration | | | | |
| K⁺-beads' Effect on | 9.23 | 7.17 | 11.02 | 10.51 |
| K⁺ in the Culture | | | | |
| Medium (mM) | | | | |
| K⁺-beads' Effect on | 130.43 | 115.94 | 121.74 | 121.74 |
| Na⁺ in the Culture | | | | |
| Medium (mM) | | | | |

### Embodiment 1-2: Comparative Ion Exchange Performance of the K+-Beads with Different Composition Ratios

To compare the ion exchange performance of the K+-beads with different composition ratios, four distinct hydrogel bead formulations were designed. The composition ratios of the K+-beads, depicted in A to D in FIG. 3, are presented in Table 2.

TABLE 2
Formulation of the K+-beads with Varying Compositional Ratios.

| | Potassium-alginate | SrCl₂ | KCl |
|---|---|---|---|
| A in FIG. 3 | 2.5 wt % | 3.0 wt % | 0.0 wt % |
| B in FIG. 3 | 2.5 wt % | 3.0 wt % | 1.2 wt % |
| C in FIG. 3 | 2.5 wt % | 2.5 wt % | 1.2 wt % |
| D in FIG. 3 | 3.0 wt % | 2.5 wt % | 1.5 wt % |

The ion concentrations in the culture medium are shown in A to D in FIG. 3. The dashed line represents sodium ion concentration, the thin line represents potassium ion concentration, and the bold line represents the total ion concentration. In the absence of KCl supplementation (A in FIG. 3), the total ion concentration decreases because of the disparity in the osmotic pressure between the interior and exterior of the K+-beads. This deviation does not align with the desired simulation of the physiological equilibrium of total ion concentration within a living organism. Consequently, the concentration of KCl required to mimic the concentration of NaCl in-vivo was determined and added to the K+-beads (B in FIG. 3) to mitigate the declining trend in the total ion concentration.

Reducing the quantity of SrCl2 to 2.5 wt % (C in FIG. 3) or simultaneously increasing the concentrations of KCl and potassium-alginate (D in FIG. 3) maintains the total ion concentration within an appropriate range, with the composition shown in D in FIG. 3 being preferable. In summary, the K+-beads used in the present disclosure are composed in accordance with the ratios presented in D in FIG. 3 and serve as the formulation for ion-exchange material 51 in this work.

### Embodiment 2: In-Vivo Ion-Exchange During the Growth of Simulated Cochlear Progenitor Cells (CPC)

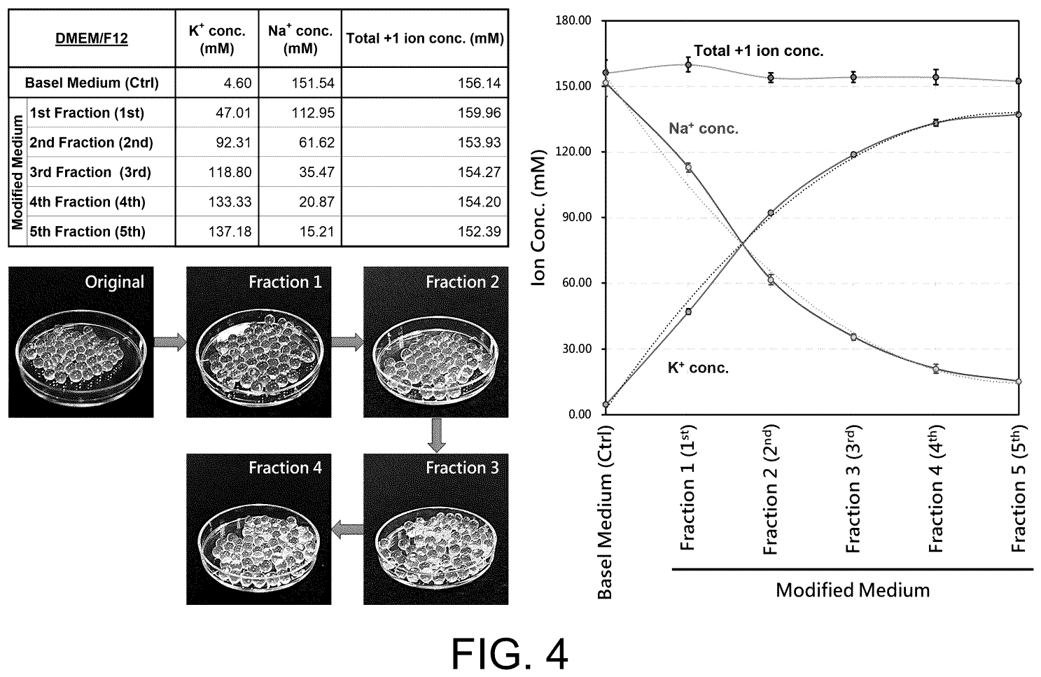

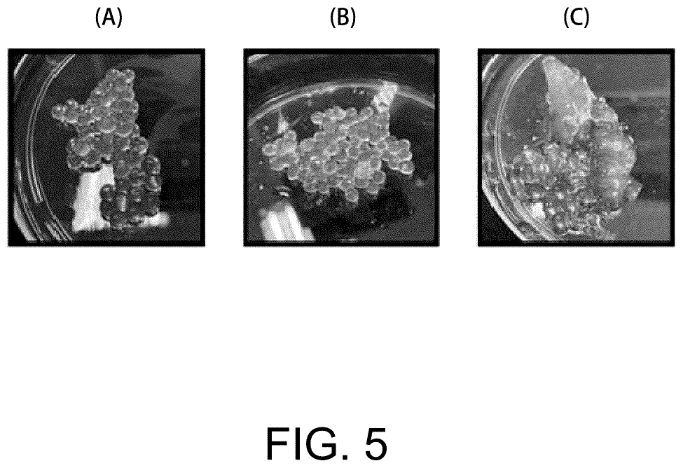

As shown in Tables 3 and 4, as well as FIG. 4, to simulate the changes in ion concentrations in the endolymph during the growth of mouse cochlear progenitor cells, a modified medium after four fractions of ion-exchange was used in comparison to the baseline culture medium DMEM/F12. The modified medium was used to cultivate mouse cochlear progenitor cells. The Na+ and K+ concentrations necessary for culturing mouse cochlear progenitor cells were adjusted according to the variations in sodium and potassium ion concentrations during maturation of the mouse cochlea. This allowed the cells to grow in an environment in which Na+ and K+ concentrations were gradually replaced within the culture medium.

TABLE 3
Formulation of the K+-beads with Varying Compositional Ratios.

| | | | | Total Ion |
|---|---|---|---|---|
| | | K⁺ (mM) | Na⁺ (mM) | Conc. (mM) |
| Basal Medium | | 4.09 | 153.60 | 157.69 |
| Modified | Fraction 1 | 46.15 | 114.28 | 160.43 |
| Medium | Fraction 2 | 92.31 | 60.24 | 152.55 |
| | Fraction 3 | 115.38 | 39.75 | 155.13 |
| | Fraction 4 | 130.77 | 24.22 | 150.99 |
| | Fraction 5 | 135.89 | 14.90 | 150.79 |

TABLE 4
Variation in the Concentration of Endolymph
within the Murine Cochlea.

| | | | Modified | Basel |
|---|---|---|---|---|
| | K⁺ | Na⁺ | Medium | Medium |
| | (mM) | (mM) | (ml) | (ml) |
| E14.5-15.5 | 8 | 150 | 0.030 | 0.970 |
| E15.5-16.5 | 12 | 146 | 0.061 | 0.939 |
| E16.5-17.5 | 16 | 142 | 0.092 | 0.908 |
| E17.5-18.5 | 20 | 138 | 0.123 | 0.877 |
| E18.5-19.5 | 30 | 128 | 0.200 | 0.800 |
| P1 | 70 | 88 | 0.510 | 0.490 |
| P2 | 85 | 73 | 0.626 | 0.374 |
| P3 | 100 | 58 | 0.742 | 0.258 |
| P4 | 115 | 43 | 0.858 | 0.142 |
| P5 | 130 | 28 | 0.974 | 0.026 |

### Embodiment 3: Preservation of the K+-Beads

As illustrated in A to C in FIG. 5, this study compared the effectiveness of three different solutions/preservation fluids for the preservation of alginate-potassium hydrogel beads. These solutions included deionized water (A in FIG. 5), 150 mM KCl solution (B in FIG. 5), and potassium-alginate solution used for bead preparation (C in FIG. 5). As shown in Table 5, storing the K+-beads in low-osmolarity deionized water (A in FIG. 5) resulted in ion diffusion from the K+-beads into the preservation fluid, leading to a decrease in ion concentration within the K+-beads. However, when the K+-beads were stored in either 150 mM KCl solution (B in FIG. 5) or potassium-alginate solution (C FIG. 5), they maintained effective ion exchange. Nevertheless, the K+-beads preserved in potassium-alginate solution exhibited competitive interactions between Sr2+ on the K+-bead surface and the preservation fluid, making it difficult to separate the K+-beads from the preservation fluid (C in FIG. 5).

TABLE 5
Variations in Na+ and K+ within the Culture Medium Due to the
Storage of the K+-beads in Different Storage Solutions.

| | | K⁺ (mM) | | |
|---|---|---|---|---|
| | | | 150 mM | |
| | | Deionized | KCl | Potassium-alginate |
| Storage Solutions | | Water | Solution | Solution |
| Modified | Fraction 1 | 35.89 | 45.14 | 47.89 |
| Medium | Fraction 2 | 50.17 | 90.58 | 93.83 |
| | Fraction 3 | 61.04 | 120.16 | 121.05 |
| | Fraction 4 | 65.17 | 127.73 | 130.68 |
| | Fraction 5 | 67.58 | 130.82 | 132.45 |

### Embodiment 4: Removal of Nitrogen-Containing Metabolic Wastes with the K+-Beads

As depicted in A in FIG. 6, the K+-beads were capable of adsorbing ammonium ions (NH4+) to facilitate the removal of nitrogen-containing metabolic wastes from the environment. When a test strip was introduced into a culture medium that had been enriched with nitrogen-containing metabolic waste and cultured cells, it exhibited a deep blue color, as shown in B in FIG. 6.

As illustrated in C to F in FIG. 6 and Table 6, after a single treatment with the K+-beads in the culture medium for 10 min, followed by the insertion of a test strip (C in FIG. 6), the color of the test strip was lighter than that in B in FIG. 6. With repeated ion exchange (adsorption of NH4+) using the fresh K+-beads in the culture medium for two to four cycles (D to F in FIG. 6), the test strip color gradually became lighter, concomitant with a decrease in NH3/NH4+ ion concentration within the culture medium.

TABLE 6
The Efficacy of the K+-beads for NH4+ Removal.

| Ion- exchange | 0 | 1 | 2 | 3 | 4 |
|---|---|---|---|---|---|
| Frequency | (B in | (C in | (D in | (E in | (F in |
| (number of times) | FIG. 6) | FIG. 6) | FIG. 6) | FIG. 6) | FIG. 6) |
| NH₃/NH₄⁺ | >6.0 (over | ~6.0 | 3.0-6.0 | 1.0-3.0 | 0.5-1.0 |
| (ppm) | detectable | | | | |
| | limitation) | | | | |

### Embodiment 5: Temporal Variation in a Bioreactor Apparatus for Ion-Exchange

As shown in A and B in FIG. 7 and Table 7, the ion-exchange substrate of the present disclosure can be combined with the disclosed bioreactor to create an environment/system capable of continuously exchanging ion concentrations and cultivating cells. Table 7 presents the test results of the K+-beads exchanging ion concentrations within the bioreactor apparatus tested at a flow rate of 1 ml/min. Equilibrium was reached after 24 h of exchange, which is similar to the results obtained in Example 2. Therefore, the speed of the pump disclosed in this work can be adjusted to control the ion concentrations within the system.

TABLE 7
Temporal Variation of K+ Concentration.

| | | Modified Medium | | | |
|---|---|---|---|---|---|
| | | Fraction 1 | Fraction 2 | Fraction 3 | Fraction 4 |
| K⁺ (mM) | 1 h | 31.73 | 60.89 | 92.33 | 117.32 |
| | 24 h | 44.89 | 89.74 | 108.97 | 125.64 |

### Embodiment 6: Biocompatibility Testing of the K+-Beads

### Embodiment 6-1: Cytotoxicity Assay of the K+-Beads

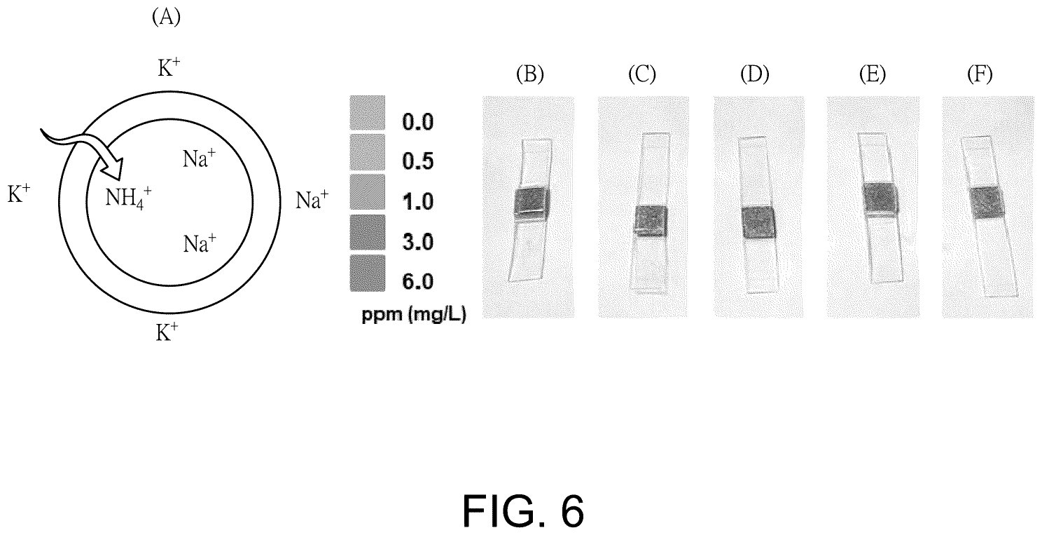

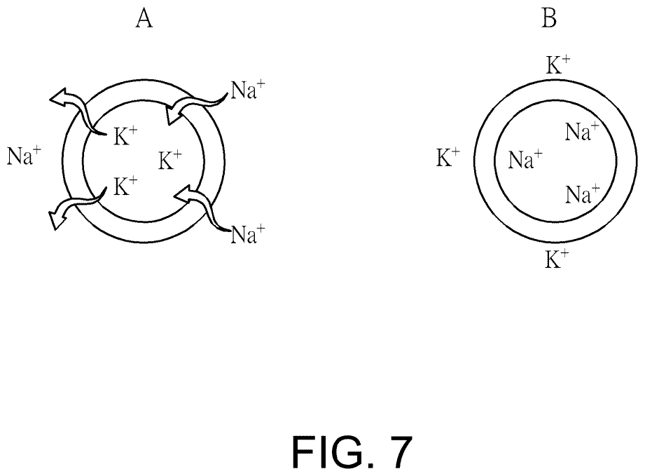

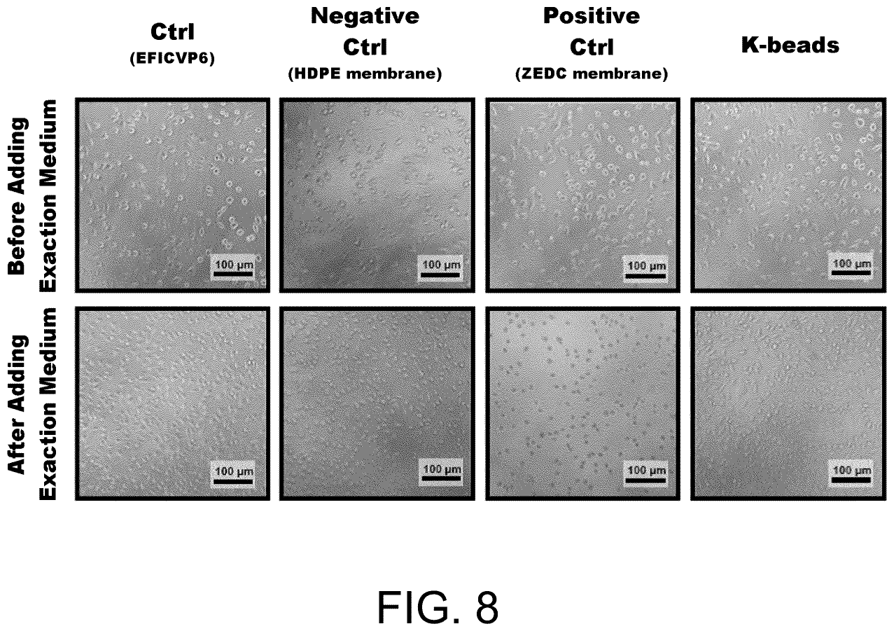

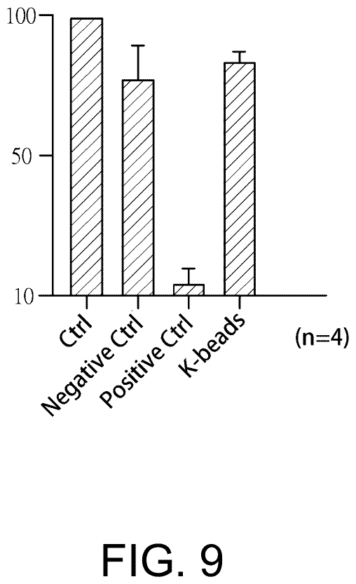

To evaluate the biocompatibility of the K+-beads, four groups were designed. The test group utilized an extract of the K+-beads as the experimental medium (condition of extract medium: the concentration of the test substance is 0.2 g/ml), the control group used a basal medium with 10% FBS (fetal bovine serum), the positive control group used an extract of the ZEDC film in medium with 10% FBS, and the negative control group used an extract of the HDPE film in medium with 10% FBS. As shown in FIG. 8, the cell growth morphology in the medium modified by the K+-beads was consistent with that of the control group. As shown in FIG. 9, the cell viability when cultured in a medium modified by the K+-beads was approximately 87%, indicating that the test substance was not cytotoxic. Therefore, the culture medium modified by the K+-beads used in the present disclosure did not exhibit cytotoxicity towards the cells.

### Embodiment 6-2: Cellular Growth and Differentiation Morphology of Mouse Cochlear Progenitor Cells (CPC) Cultured in Medium Modified by the K+-Beads

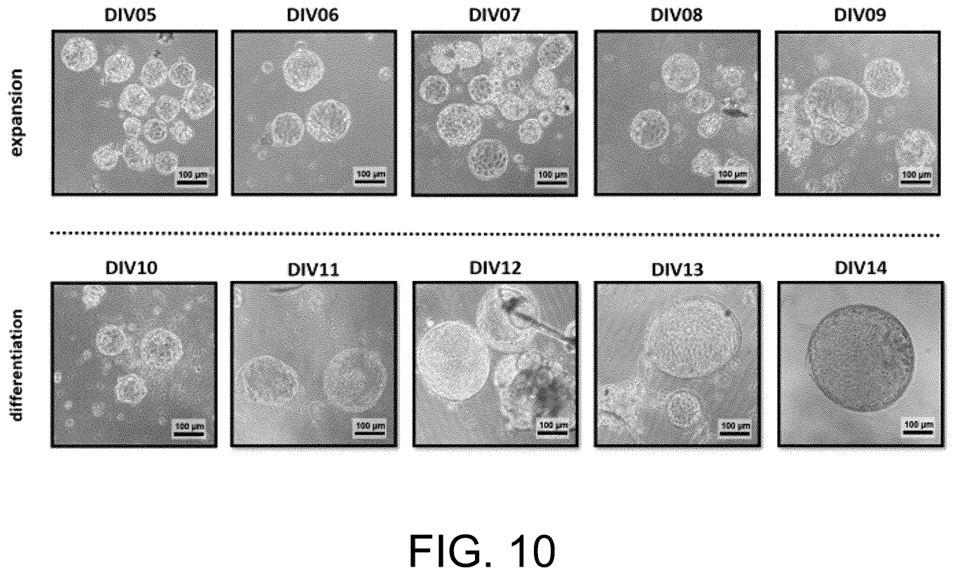

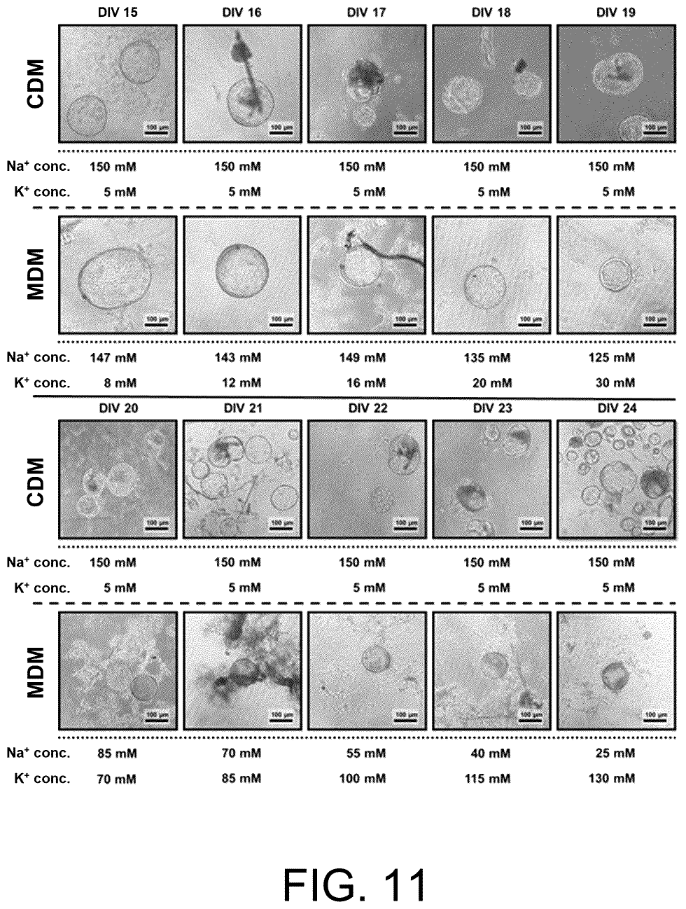

As shown in FIG. 10, during the in-vitro growth period (DIV) from DIV0 to DIV9, the size of mouse CPC clusters significantly increased. Entering the early differentiation stage from DIV10 to DIV14, some of the CPC clusters fused and continued to increase in size. As shown in FIG. 11, to compare the differentiation of mouse CPC clusters cultured in media with different ion concentrations, a modified differentiation medium (MDM) was prepared by ion exchange four times, while a standard DMEM/F12 culture medium was used as the control differentiation medium (CDM). The ion concentrations were adjusted in the MDM to mimic the changes in Na+ and K+ concentrations during cochlear maturation. The cells were cultured in an environment in which the ion concentrations were gradually replaced. DIV15 to DIV19 were considered the early stages of CPC cluster differentiation, and DIV20 to DIV24 defined the stages of maturation into hair cells. As shown in A and B in FIG. 12, immunocytochemistry (ICC) was used to compare the histological characteristics of mouse CPC cultured in the control group and the group modified by the K+-beads. Expression of MYO7A, which encodes myosin-7A, a protein found in sensory hair cells in the inner ear, was detected. Hoechst 33342 dye (blue fluorescence, Thermo Fisher Scientific) was used to stain the cell nuclei, and anti-myosin 7A antibodies (red fluorescence, Abcam) were used to label the location and distribution of MYO7A. In the control group (A in FIG. 12), MYO7A was expressed inward in the CPC clusters. Conversely, in the group modified by the K+-beads (B in FIG. 12), MYO7A was expressed in CPC clusters.

### Embodiment 6-3: Size Distribution of Mouse CPC Clusters Cultured in Medium Modified by the K+-Beads

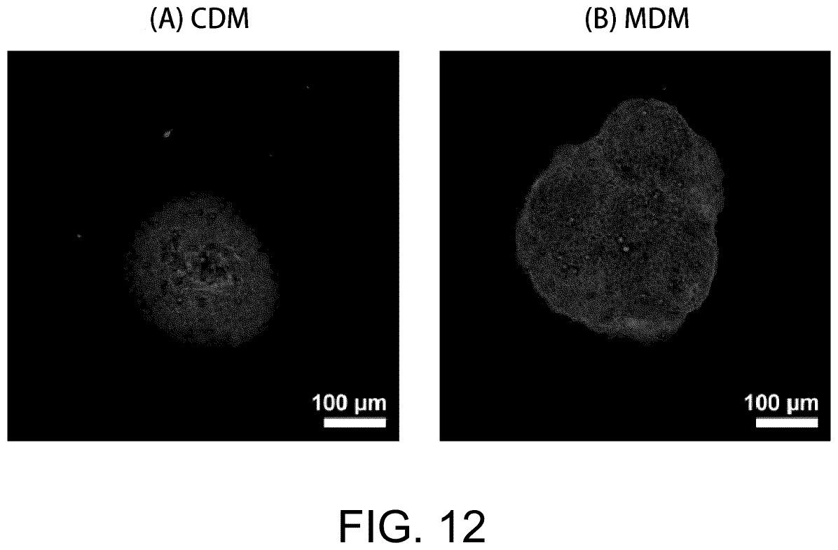

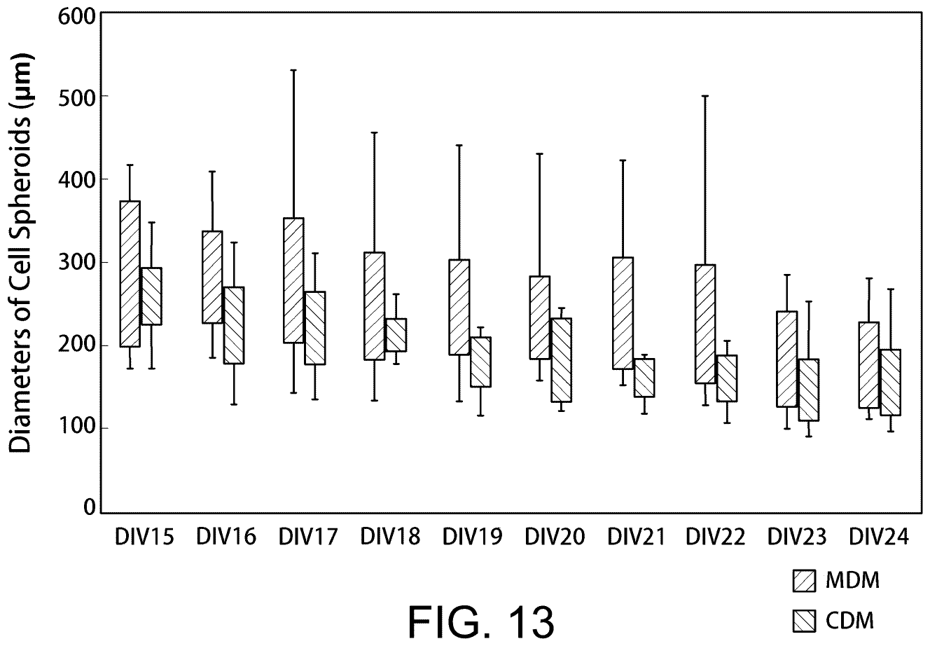

As shown in FIG. 13, a quantitative comparison was made between CPC clusters cultured in modified differentiation medium (MDM) and those cultured in control differentiation medium (CDM) during the late stages of in-vitro differentiation from DIV15 to DIV24. Although the CPC clusters in the MDM group were larger than those in the CDM group, the difference was not statistically significant. However, from DIV15, when K+ in the MDM increased to 80 mM and Na+ decreased to 77 mM, the cell clusters in the CDM group exhibited a significant decrease in size, with many CPC clusters showing damage and reduced quantity. In contrast, the trend of decreasing CPC cluster size was less pronounced in the MDM group, and CPC cluster morphology was better maintained. In summary, culturing CPC in a medium with gradual, controlled Na+/K+ exchange allowed for normal cell growth and differentiation.

### Embodiment 6-4: Live/Dead Cell Viability Assay of Mouse CPC Clusters Cultured in Medium Modified by the K+-Beads

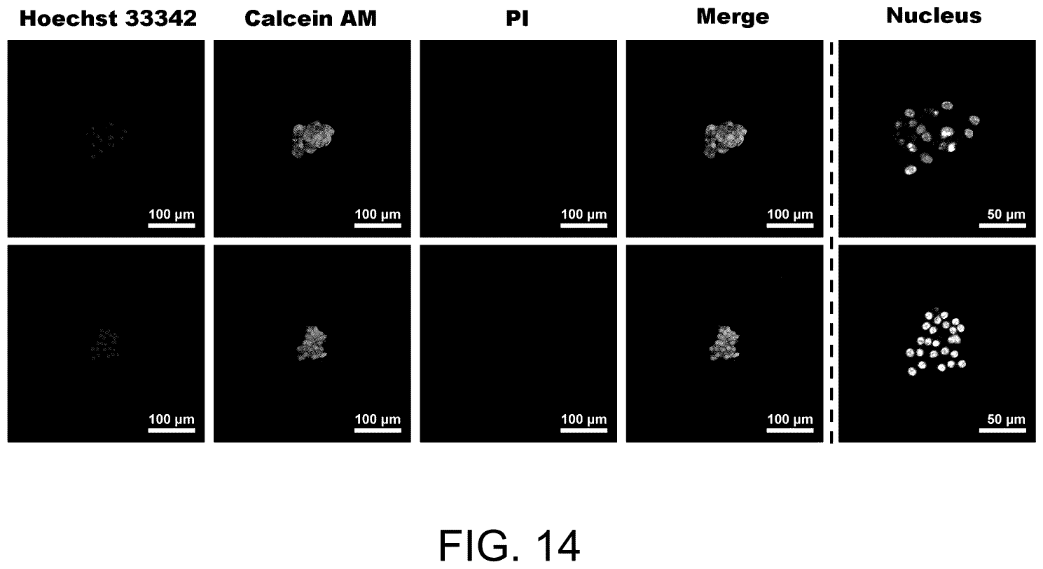

To compare the viability of cochlear progenitor cell clusters cultured in MDM and CDM, cells from both groups were stained with Calcein AM (live cell marker, green fluorescence, Thermo Fisher Scientific)/Propidium iodide (PI, dead cell marker, red fluorescence, Thermo Fisher Scientific)/Hoechst 33342 (nuclear marker, blue fluorescence, Thermo Fisher Scientific) for fluorescence observation. Calcein AM was used to label live cells, as it can pass through cell membranes and be activated by intracellular esterase to produce green fluorescence. The excitation/emission wavelengths used were 490/515 nm. PI was used to label dead cells, as it cannot penetrate intact cell membranes and nuclear membranes. Only cells with damaged membranes, indicative of dying or dead cells, allowed PI to enter the nucleus and release a red fluorescence. The excitation/emission wavelengths used were 535/615 nm. Hoechst 33342 was used to label all cells with nuclei, emitting blue fluorescence. Therefore, a combination of Calcein AM, PI, and Hoechst 33342 staining was used to determine cell viability. As shown in FIG. 14 (nucleus; dashed line on the right), the nuclei of the cells cultured in MDM were smaller (6 μm) than those of the control group cultured in CDM (10 μm). However, the improved differentiation medium group exhibits a higher cell survival rate, consistent with the well-established observation in this art that nuclei tend to shrink during hair cell development in the cochlea.

In some embodiments of the present disclosure, ion-exchange materials capable of serving as ion-exchange substrates and adsorbing nitrogen-containing metabolic wastes are created using the properties of alginates and various cations. In the specific embodiment of the disclosure, a system for ion replacement is provided, adjusting the ion concentrations and compositions of Na+ and K+ in culture media/cultivation fluids to the specific proportions required for cell culture, with this system being applied to the cultivation and differentiation of cochlear progenitor cells.

In some embodiments of the present disclosure, referring to the gradual replacement of cochlear hair cells in the inner car by a high K+/low Na+ environment during embryonic development, an in vitro environment simulating the in vivo inner car was established. Results from the cell cultures in this study indicate that cochlear progenitor cell clusters proliferating in a high Na+/low K+ environment increase in cell number and cluster size over time. In the early stages of hair cell differentiation in a high Na+/low K+ environment, there was no significant change in CPC clusters over time. In the later stages of hair cell differentiation and maturation in the high Na+/low K+ environment, both the size and number of cell clusters decreased, with MYO7A being expressed inward in the cell clusters. However, as the transition to a high K+/low Na+ environment gradually increased, the trend of decreasing cluster size became less pronounced, and the cluster configuration was better maintained, with MYO7A being expressed outwardly in the CPC clusters.

In some embodiments of the disclosure, cell staining results in cell viability detection show that cell nuclei in cell clusters cultured in a high Na+ environment are larger than those in cell clusters cultured in a high K+ environment, and cell arrangement is denser, consistent with the known phenomenon in the field of cochlear hair cell differentiation, in which cell nuclei tend to shrink during the process.

The ion-exchange/replacement method developed in this work can not only be applied to the proliferation, differentiation, and maturation of inner ear hair cells but also to in vitro testing models with ion concentration adjustment requirements. Furthermore, they can be applied as cell-based in vitro proliferation, differentiation, and maturation culture platforms for hearing loss therapy. Ion-exchange materials capable of serving as ion-exchange substrates and adsorbing nitrogen-containing waste were created using the properties of alginates and various cations. In certain disclosed embodiments, a system for ion replacement is provided, adjusting the ion concentrations and compositions of Na+ and K+ in culture media/cultivation fluids to the specific proportions required for cell culture, with this system being applied to the cultivation and differentiation of CPC

In some embodiments of the disclosure, referring to the gradual replacement of cochlear hair cells in the inner ear by a high K+/low Na+ environment during embryonic development, an in-vitro environment simulating the in-vivo inner car was established. The results from cell cultures in this study indicate that CPC clusters proliferating in a high Na+/low K+ environment increase in cell number and cluster size over time. In the early stages of hair cell differentiation in the high Na+/low K+ environment, there is no significant change in CPC clusters over time. In the later stages of hair cell differentiation and maturation in the high Na+/low K+ environment, both the size and number of cell clusters decrease, with MYO7A being expressed inwardly in the cell clusters. However, as the transition to a high K+/low Na+ environment gradually increased, the trend of decreasing CPC cluster size was less pronounced, and the cluster configuration was better maintained, with MYO7A being expressed outwardly in the cell clusters.

In some embodiments of the disclosure, cell staining results in cell viability detection show that cell nuclei in CPC clusters cultured in a high Na+ environment are larger than those in cell clusters cultured in a high K+ environment, and cell arrangement is denser, consistent with the known phenomenon in the field of cochlear hair cell differentiation, in which cell nuclei tend to shrink during the process.

The ion-exchange/replacement method developed in this work can not only be applied to the proliferation, differentiation, and maturation of inner ear hair cells, but also to in-vitro testing models with ion concentration adjustment requirements. Furthermore, they can be applied to cell-based in vitro proliferation, differentiation, and maturation culture platforms for hearing loss therapy.

### Embodiment 7: Establishment of an In-Vitro Model for Cochlea-Like Hair Cell Proliferation, Differentiation, and Maturation Using Mouse CPC-Laden Scaffold

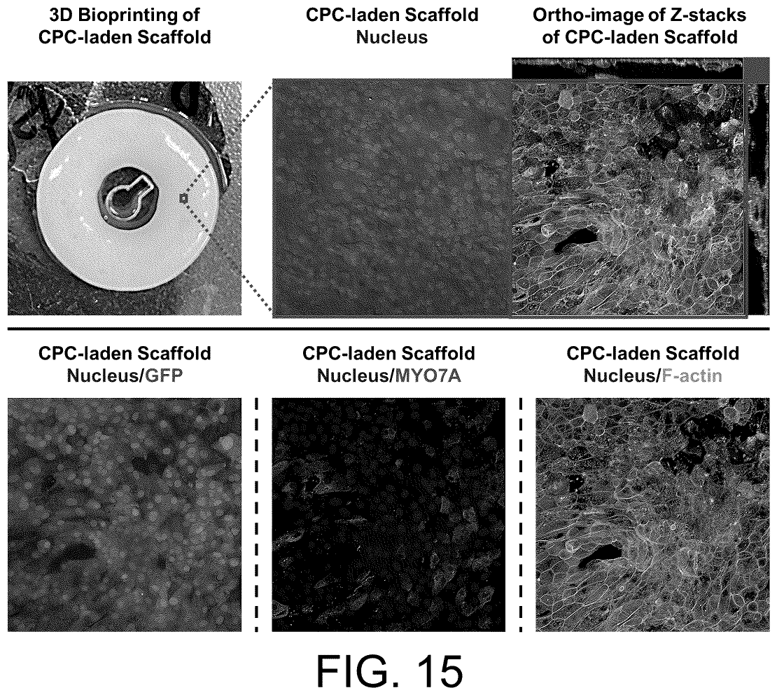

To ensure the application of a scaffold for placing mouse CPC on the culture-dish holder 43 and for in-vitro differentiation of cochlea-like hair cells using a bioreactor device, as illustrated in FIG. 15, this disclosure involves establishing an in-vitro environment within cultivation chamber 4, utilizing the scaffold 42 (Collgel®, but not limited thereto). This environment promotes the proliferation, differentiation, and maturation toward inner ear hair cells. These CPC were labeled using Hoechst 33342 (nuclear staining, stained in blue)/GFP (green fluorescent protein, labeling all cytoplasm)/MYO7A (myosin VIIa, labeling inner ear hair cells, shown in red)/F-actin (labeled F-actin, represented in yellow) for confocal microscopy Z-axis orthogonal image observation.

In summary, the combination of GFP, MYO7A, F-actin, and Hoechst 33342 dyes can be employed to determine the feasibility of applying scaffolds for the 3D in-vitro cultivation of CPC toward inner ear hair cell differentiation. Successfully differentiated cochlea-like hair cells were primarily located and distributed on the surface of the scaffold.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A bioreactor apparatus, comprising:
a liquid storage chamber for storing a liquid comprising a first ion;
a pump;
a cultivation chamber for accommodating the liquid and a cell to be cultured; and
an ion-exchange chamber containing an ion-exchange substrate comprising a second ion,
wherein affinity of the second ion to the ion-exchange substrate is lower than affinity of the first ion to the ion-exchange substrate, or molar concentration of the second ion is higher than molar concentration of the first ion; and
wherein the liquid storage chamber, the pump, the cultivation chamber, and the ion-exchange chamber are connected by a pipeline to form a closed loop, and the pump is configured to provide pressure to drive flow of the liquid in the closed loop.

2. The bioreactor apparatus of claim 1, wherein the ion-exchange substrate has a semi-permeable membrane encapsulating the liquid comprising the second ion, thereby allowing the first ion to enter the ion-exchange substrate and releasing the second ion into the liquid, and wherein the ion-exchange substrate is formed from a crosslinked construct of an alginate and a third ion, and affinity of the third ion to the ion-exchange substrate is greater than affinity of the second ion to the ion-exchange substrate.

3. The bioreactor apparatus of claim 2, wherein the first ion comprises Na⁺, NH₄⁺, or a heavy metal ion; wherein the second ion comprises Na⁺, K⁺, Ca2⁺, or NH₄⁺; and wherein the third ion comprises a divalent cation, a trivalent cation, or a polyvalent cation.

4. The bioreactor apparatus of claim 3, wherein the heavy metal ion is at least one selected from a group consisting of Cd²⁺, Pb²⁺, Cr²⁺, Sb²⁺, Hg²⁺, Fe²⁺, Co²⁺, Zn²⁺, Ni²⁺, Cu²⁺, Tb³⁺, Dy³⁺, Fe³⁺, and A¹³⁺; the divalent cation is at least one selected from a group consisting of Sr²⁺, Ca²⁺, Cd²⁺, Pb²⁺, Cr²⁺, Ba²⁺, Se²⁺, Sb²⁺, Hg²⁺, Mg²⁺, Fe²⁺, Co²⁺, Zn²⁺, Ni²⁺, As²⁺, and Cu²⁺; and the trivalent cation is at least one selected from a group consisting of Tb³⁺, Dy³⁺, Fe³⁺, and Al³⁺.

5. The bioreactor apparatus of claim 4, wherein the third ion is Sr²⁺, Ca²⁺ or Mg²⁺.

6. The bioreactor apparatus of claim 5, wherein the second ion is K⁺, and the third ion is Sr²⁺.

7. The bioreactor apparatus of claim 1, wherein the crosslinked construct forms a cavity having the semi-permeable membrane.

8. The bioreactor apparatus of claim 7, wherein the crosslinked construct is in a form of a particles or a sphere, and the particle or the sphere has a diameter of from 0.5 mm to 50 mm.

9. A method for regulating ion concentration in a liquid, comprising:
providing an ion-exchange substrate comprising a second ion and having a semi-permeable membrane enclosing a liquid comprising the second ion; and
disposing the ion-exchange substrate in the liquid comprising a first ion to perform ion exchange, comprising:
allowing the first ion to enter the ion-exchange substrate through the semi-permeable membrane; and
releasing the second ion from the ion-exchange substrate into the liquid through the semi-permeable membrane,
wherein affinity of the second ion to the ion-exchange substrate is lower than affinity of the first ion to the ion-exchange substrate, or molar concentration of the second ion is higher than molar concentration of the first ion.

10. The method of claim 9, wherein the liquid further comprises the second ion, the concentration of the second ion in the ion-exchange material is greater than the concentration of the second ion in the liquid during the ion exchange; and subsequent to ion exchange, the concentration of the first ion in the liquid decreased, and the concentration of the second ion in the liquid increased.

11. The method of claim 9, wherein the ion-exchange material is a crosslinked construct of alginate with a third ion, and affinity of the third ion to the ion-exchange substrate is greater than affinity of the second ion to the ion-exchange substrate.

12. The method of claim 11, wherein the first ion comprises Na⁺, NH₄⁺, or a heavy metal ion; wherein the second ion comprises Na⁺, K⁺, Ca²⁺, or NH₄⁺; and wherein the third ion comprises a divalent cation, a trivalent cation, or a polyvalent cation.

13. The method of claim 12, wherein the heavy metal ion is at least one selected from the group consisting of Cd²⁺, Pb²⁺, Cr²⁺, Sb²⁺, Hg²⁺, Fe²⁺, Co²⁺, Zn²⁺, Ni²⁺, Cu²⁺, Tb³⁺, Dy³⁺, Fe³⁺, and Al³⁺; the divalent cation is at least one selected from the group consisting of Sr²⁺, Ca²⁺, Cd²⁺, Pb²⁺, Cr²⁺, Ba²⁺, Se²⁺, Sb²⁺, Hg²⁺, Mg²⁺, Fe²⁺, Co²⁺, Zn²⁺, Ni²⁺, As²⁺ and Cu²⁺; and the trivalent cation is at least one selected from the group consisting of Tb³⁺, Dy³⁺, Fe³⁺, and Al³⁺.

14. The method of claim 13, wherein the third ion is Sr²⁺, Ca²⁺ or Mg²⁺.

15. The method of claim 12, wherein the second ion is K⁺, and the third ion is Sr²⁺.

16. The method of claim 11, wherein the crosslinked construct forms a cavity having the semi-permeable membrane.

17. The method of claim 16, wherein the crosslinked construct is in a form of a particle or a sphere, and the particle or the sphere has a diameter of from 0.5 mm to 50 mm.

18. A method for culturing a cell, comprising:
providing a bioreactor apparatus comprising:
a liquid storage chamber for storing a culture medium comprising a first ion;
a pump;
a cultivation chamber for accommodating the culture medium and the cell to be cultured; and
an ion-exchange chamber containing an ion-exchange substrate comprising a second ion,
wherein affinity of the second ion to the ion-exchange substrate is lower than affinity of the first ions to the ion-exchange substrate, or molar concentration of the second ions is significantly higher than molar concentration of the first ions; and
wherein the liquid storage chamber, the pump, the cultivation chamber, and the ion-exchange chamber are connected by a pipeline to form a closed loop;
disposing the culture medium and the cells in the cultivation chamber;
driving the culture medium to flow in a closed loop by the pump; and
performing ion exchange by the ion-exchange substrate to decrease concentration of the first ion in the culture medium and increase concentration of the second ion in the culture medium.

19. The method of claim 18, wherein the first ion is Na⁺, NH₄⁺, heavy metal ions, or any combination thereof, and the second ion is K⁺.

20. The method of claim 18, wherein the cell is a hair cell of an inner ear.
